(19) United States
(12) Patent Application Publication
(10) Pub. No.: US 2017/0368018 A1
(43) Pub. Date: Dec. 28, 2017

(54) USE OF ELLAGIC ACID DIHYDRATE IN PHARMACEUTICAL FORMULATIONS TO REGULATE BLOOD GLUCOSE LEVELS

(71) Applicant: HALO LIFE SCIENCE, LLC, Victoria, TX (US)

(72) Inventor: Michael REYES, Victoria, TX (US)

(73) Assignee: HALO LIFE SCIENCE, LLC, Victoria, TX (US)

(21) Appl. No.: 15/536,033

(22) PCT Filed: Dec. 4, 2015
(86) PCT No.: PCT/US15/63909

§ 371 Date: Jun. 14, 2017

Related U.S. Application Data
(60) Provisional application No. 62/094,746, filed on Dec. 19, 2014.

(51) Int. Cl. A61K 31/35; C07D 311/02; A61K 36/73; A61K 36/45; A61K 36/185; C07D 493/06; A61K 31/05

(52) U.S. Cl. CPC A61K 31/35; C07D 493/06; C07D 311/02; A61K 31/05; A61K 36/45; A61K 36/185; A61K 36/73

(57) ABSTRACT

The present invention relates generally to pharmaceutical compositions and methods of use that include an ellagic acid dihydrate compound that assists in increasing glucose uptake.

### CROSS-REFERENCE TO RELATED APPLICATIONS

This application claims benefit to U.S. Provisional Patent Application No. 62/094,746 titled “USE OF ELLAGIC ACID DIHYDRATE IN PHARMACEUTICAL FORMULATIONS TO REGULATE BLOOD GLUCOSE LEVELS”, to Michael Reyes, filed Dec. 19, 2014. The entire contents of the referenced application are incorporated herein by reference.

### BACKGROUND OF THE INVENTION

### A. Field of the Invention

The present invention relates to formulations containing ellagic acid dihydrate and related compounds and uses thereof. More particularly, it concerns methods and formulations capable of treating or preventing hyperglycemic disorders, obesity, and metabolic conditions and disorders and the related pathologies, conditions, causes, and symptoms of such disorders and conditions.

### B. Description of Related Art

Dysregulation of glucose uptake and glucose metabolism is a growing worldwide health concern. High glucose levels in the plasma in the body (hyperglycemia) has been associated with excessive weight, obesity, Type I diabetes, Type II diabetes, gestational diabetes, latent autoimmune diabetes of adults, prediabetes, and metabolic syndrome and many other diseases or secondary diseases. Obesity, diabetes, prediabetes, and metabolic syndrome are not the only disease that are correlated with weigh gain, dysregulation of glucose metabolism, or higher than normal glucose levels. Such conditions have been correlated with other diseases and pathologies such as neuropathologies, Alzheimer's, cardiovascular diseases, and cancers.

Obesity is a complex disorder involving an excessive amount of body fat. Generally, a body mass index (BMI) of over 30 is considered obese. Strong links between obesity and chronic diseases, such as diabetes, cardiovascular disease, cerebrovascular disease, and many cancers, are well-established. Further, obesity contributes to the economic costs to governments, healthcare systems, and individuals and decreases individual's quality of life. Diabetes is a serious health problem in modern society that is linked to obesity; however, diabetes does not require obesity. Diabetes presents as persistent hyperglycemia (high glucose levels). Agents that reduce glucose levels by decreasing insulin resistance, increase glucose uptake, or stimulating insulin secretion are being investigated. Diabetes can be a major contributor to heart disease, stroke, neuropathy, ulcers in extremities, poor blood circulation and peripheral nerve dysfunction, and lead to kidney failure and/or blindness.

Subjects with Type 1 diabetes produce little to no insulin, and thus the primary treatment for Type 1 diabetes is insulin therapy. Subjects diagnosed with Type II diabetes produce insulin, but their bodies do not use it effectively (e.g., cells in the body become desensitized to insulin or do not respond to insulin). The condition is referred to as being insulin resistant. Subjects with gestational diabetes become insulin resistant during pregnancy and such insulin resistance increases the health risk to both the mother and child and increases the risk of birth defects. Due to limitations of non-insulin treatments or non-compliance with treatment regiments, many subjects with Type II diabetes eventually present with chronic hyperglycemia. Such chronic hyperglycemia can desensitize the insulin secreting pancreatic β cells to glucose, causing decreased secretion of insulin in response to glucose. Thus, many subjects with Type II diabetes eventually require insulin therapy, and because of insulin resistance, the treatment may not be effective. A typical insulin regimen involves administering several injections of insulin each day: long-acting basal insulin one or two times per day and rapid-acting insulin at mealtimes. Although this treatment regimen is accepted as effective, it has limitations due to the inconvenience and pain of self-injection. As a result, patients tend not to comply adequately with the prescribed treatment regimens. In many cases, Type II diabetes and gestational diabetes can be treated with oral drugs therapy, but these therapies are limited. Many of current formulations are designed to stimulate the beta cells of the pancreas to release more insulin, to decrease the amount of glucose produced by the liver, inhibit DPP-4 production in the body, inhibit sodium-glucose transporter 2 function in the kidneys, or inhibit alpha-glycosidase production. Many of these drugs are used in combination to manage the blood glucose levels of a subject. These oral therapies, however, are not used to treat Type 1 diabetes. Thus, a single treatment available for both Type I and Type II diabetes is not currently available.

Prediabetes is a condition where a person has blood sugar levels higher than normal, but not high enough to be diagnosed with diabetes. Subjects with prediabetes have a higher risk for developing Type II diabetes, heart disease, stroke, and other serious health problems. The current treatment for prediabetes is weight management through diet and an increase in physical activity.

Metabolic syndrome is the major contributor to the development of diabetes and is related to an increased risk of cardiovascular disease. Diagnosis of metabolic syndrome requires central obesity plus any two of the following four factors: elevated triglycerides; reduced HDL-cholesterol; hypertension; or abnormal fasting plasma glucose. Currently, metabolic syndrome is treated by control or treatment of the underlying factors, such as abdominal obesity, atherogenic dyslipidemia, raised blood pressure, insulin resistance with or without glucose intolerance, proinflammatory state, and/or prothrombotic state.

Alternative methods to treat many types of cancers and diseases such as diabetes include the use of plant and fruit extracts (for example, pomegranate juice, strawberries, blueberries). These extracts include many antioxidant compounds, for example, the polyphenol ellagitannins. International Application Publication No. WO 2012/088519 to Rinsch et al. describes that the combination of ellagitannins, free ellagic acid, and urolithins from pomegranate juice lowers fasting glucose plasma levels in mice.

### SUMMARY OF THE INVENTION

The present invention provides a solution to the current problems facing regulation of blood glucose levels and weight management. It was surprisingly found that ellagic acid dihydrate provided an increase in glucose uptake per unit dose as compared to free ellagic acid under the same in vitro conditions. Notably, ellagic acid showed little to no glucose uptake per unit dose. It was also surprisingly found that the ellagic acid dihydrate induced glucose uptake can be similar to or better than a glucose uptake profile for insulin under the same conditions while free ellagic acid had a different glucose uptake profile. Without wishing to be bound by theory, it is believed that ellagic acid inhibits Casein Kinase 2 (CK2) activity, thereby allowing fat cells to burn energy (e.g., allowing white fat to turn into brown fat). From this data and from data that normal and diabetic mice treated with free ellagic acid experience weight loss, it is believed that treatment with ellagic acid dihydrate would provide an even greater weight loss effect. The compositions described throughout this specification can be formulated for use to control, treat, or possibly prevent hyperglycemia and/or excessive weight, or are used to prevent, control, or treat Type I diabetes, Type II diabetes, gestational diabetes, latent auto-immune diabetes of adulthood, insulin resistant diseases, prediabetes, clinical obesity, metabolic syndrome, liver disease, kidney disease, coronary heart disease, cognitive diseases, or diseases associated with advanced glycation end products, or any combination thereof. Advanced glycation end products diseases can include atherosclerosis, chronic renal failure, dementia, or Alzheimer's. In some instances, use of the ellagic acid dihydrate can improve liver function, improve kidney function, reduce plaque in the arteries, increase activity levels and/or cellular energy levels (e.g., by increasing levels of ATP, phosphocreatine, etc.), and the diseases and conditions associated with such functions, as these functions, diseases, and conditions can be linked to above-normal glucose concentration in the plasma in the body.

In one aspect, there is disclosed a composition formulated for administration to a subject, the composition can include an ellagic acid dihydrate compound, where the ellagic acid dihydrate compound provides an increase in glucose uptake under in vitro conditions as compared to free ellagic acid under the same conditions. The ellagic acid dihydrate compound can include one or more polyphenols, preferably a diphenol compound comprising at least two carboxylic groups or two lactones. In some embodiments, the ellagic acid dihydrate compound can include minimal amounts of tannins (for example, ellagic acid tannin), free ellagic acid or any combination thereof. The ellagic acid dihydrate can be represented by the chemical structure:

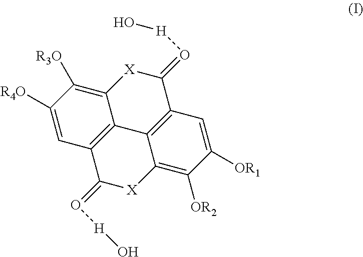

Where R₁, R₂, R₃, and R₄ are each independently selected from a hydrogen (H), a C₁-C₂₀ alkyl or ether group, an acyl group, an aryl group, an aralkyl group, an amide, an amino acid, and a heterocyclic group and X is a heteroatom.

The C₁-C₂₀ alkyl or ether group can include any branched and/or straight chain group. Non-limiting examples of alkyl groups include methyl, ethyl, or propyl. Ether groups can include polyethylene glycol (PEG), polypropylene glycol, or mixtures thereof. The aryl group can include a phenyl moiety and derivatives thereof. A non-limiting example of a phenyl derivative can include ortho, meta, or para, tolyl groups (C₇H₇), dimethyl benzyl groups (C₉H₉), or the like. The aralkyl group can include a benzyl group (for example, CH₂C₆H₅) and derivatives thereof. Amides can include C₁ to C₅ amides. Amino acids can include β-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, d-amino acids, n-methyl amino acids. Amino acids are commercially available. A non-limiting example of an amino acid supplier is Sigma-Aldrich®. The heteroatom can include oxygen, nitrogen or phosphorous, with oxygen being preferred. In a preferred aspect of the invention, the ellagic acid dihydrate is represented by the chemical structure:

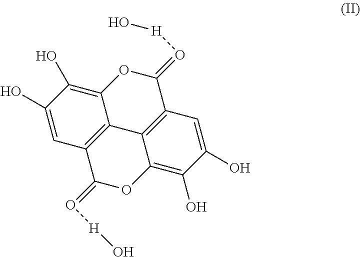

The ellagic acid dihydrate can have a purity of 95 wt. % or more, or preferable 98 wt. % or more as determined using High Pressure Liquid Chromatograph (HPLC). The ellagic acid dihydrate can have 10 wt. % or less, 5 wt. % or less, 3 wt. % or less, 2 wt. % or less of water, 5 wt % or less of tannins, 5 wt. % or less of other polyphenols. In a particular aspect, the ellagic acid dihydrate compound crystal structure does not include a co-crystal former that includes pharmaceutically acceptable carbohydrates, amines, amides, sulfonamides, carboxylic acids, sulfonic acids, phenols, polyphenols, aromatic heterocycles, xanthines and alcohols such as sucrose, fructose, and lactose. The composition can include 0.001 wt. % or more, 0.1 wt. % or more, 10 wt. % or more, or 99.0 wt. % of the ellagic dihydrate compound.

Also disclosed is a method of treating or preventing diabetes in a subject, the method comprising administering any one of the pharmaceutical compositions of the present invention to the subject. Further, there is disclosed a method of administering any one of the pharmaceutical compositions of the present invention to a subject, the method comprising administering any one of the pharmaceutical compositions of the present invention to the subject. In a particular instance, the subject has been diagnosed with Type I diabetes, Type II diabetes, gestational diabetes, latent auto-immune diabetes of adulthood, insulin resistant diseases, prediabetes, clinical obesity, metabolic syndrome, liver disease, kidney disease, coronary heart disease, cognitive diseases, diseases associated with advanced glycation end products. In another particular instance the subject can have prediabetes. In yet another particular instance, the subject has been diagnosed with metabolic syndrome. In another particular instance, the subject treated experiences an increase in cellular energy levels. Advanced glycation end product diseases can include advanced atherosclerosis, chronic renal failure, dementia, and Alzheimer's. There is also disclosed a method of treating a subject comprising administering to the subject an effective amount of an ellagic acid dihydrate compound wherein the ellagic acid dihydrate compound provides an increase in the ratio of AKT to phosphorylated AKT in a cell. Further, there is disclosed a method of treating a subject comprising administering to the subject an effective amount of an ellagic acid dihydrate compound wherein the ellagic acid dihydrate compound provides a decrease in the ratio of p44/42 MAPK to phosphorylated p44/42 MAPK in a cell. There is disclosed a method of treating a subject comprising administering to the subject an effective amount of an ellagic acid dihydrate compound wherein the ellagic acid dihydrate compound provides an increase in ruffling of filopodia at the surface of the cell. There is also disclosed a method of treating a subject comprising administering to the subject an effective amount of an ellagic acid dihydrate compound wherein the ellagic acid dihydrate compound provides an increase in GLUT4 concentration at the cellular membrane. In some embodiments, the composition is administered as part of a regime to prevent birth defects in pregnant women or women of childbearing age. The subject can be a mammal. The mammal can be a mouse, rat, rabbit, cat, dog, pig, monkey, or ape. In a preferred aspect of the invention, the mammal can be a human. Any suitable dosage of ellagic acid dihydrate compound can be formulated in the formulations of the present invention. In some aspects of the invention, a dosage of 0.5 mg/kg, 1 mg/kg, 2 mg/kg, 3 mg/kg, 4 mg/kg, 5 mg/kg or 0.5 to 5 mg/kg of the composition can decrease the maximum mean glucose concentration in the plasma of a subject. In one instance the maximum mean glucose concentration in the plasma can be reduced from 200 mg/dL or more to about 160 mg/dL or less, or 90 to 160 mg/dl, 85 mg/dL to 150 mg/dL, or 80 mg/dL to 100 mg/dL.

Also disclosed are the following embodiments 1 to 56 of the present invention. Embodiment 1 is a composition formulated for administration to a subject, the composition comprising an ellagic acid dihydrate compound, wherein the ellagic acid dihydrate compound provides an increase in glucose uptake per unit dose under in vitro conditions as compared to free ellagic acid. Embodiment 2 is the composition of embodiment 1, wherein the ellagic acid dihydrate compound comprises one or more polyphenols, preferably, a diphenol compound comprising at least two carboxylic groups or a diphenol compound having at least two lactones. Embodiment 3 is the composition of any one of embodiments 1 to 2, wherein the ellagic acid dihydrate compound is derived from a source selected from the group consisting of red raspberries, pomegranate, strawberries, and blueberries, and any combination thereof. Embodiment 4 is the composition of any one of embodiments 1 to 2, wherein the ellagic acid dihydrate compound is derived from a Punica granatum (pomegranate) and has an ellagic acid purity of 98% or more. Embodiment 5 is the composition of any one of embodiments 1 to 2, wherein the ellagic acid dihydrate compound is derived from a tree bark extract. Embodiment 6 is the composition of any one of embodiments 1 to 5, wherein the ellagic acid dihydrate compound is represented by the formula:

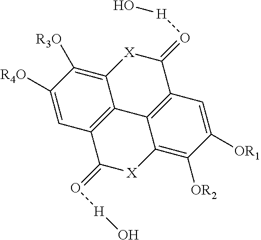

where R₁, R₂, R₃, and R₄ are each independently selected from a hydrogen (H), a C₁-C₂₀ alkyl or ether group, an acyl group, an aryl group, an aralkyl group, an amide, an amino acid, and a heterocyclic group, and X is a heteroatom. Embodiment 7 is the composition of embodiment 6, wherein the C₁-C₂₀ alkyl group is a methyl, ethyl, or propyl. Embodiment 8 is the composition of any one of embodiments 6 to 7, wherein the aryl group is a phenol moiety or derivatives thereof. Embodiment 9 is the composition of any one of embodiments 6 to 7, wherein the aralkyl group is a benzyl group or derivatives thereof. Embodiment 10 is the composition of any one of embodiments 6 to 7, wherein the heteroatom is an oxygen, nitrogen, or phosphorus. Embodiment 11 is the composition of embodiment 10, wherein the heteroatom is oxygen. Embodiment 12 is the composition of any one of embodiments 1 to 5, wherein the ellagic acid dihydrate compound consists essentially of:

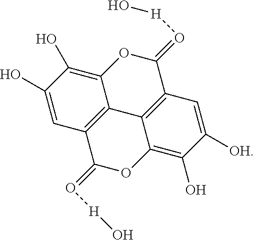

Embodiment 13 is the composition of any one of embodiments 1 to 12, wherein the ellagic acid dihydrate compound has purity of 95% or more, 98% or more, or 99% or more. Embodiment 14 is the composition of any one of embodiments 1 to 13, wherein the composition further comprises a pharmaceutical acceptable carrier and/or diluent. Embodiment 15 is the composition of embodiment 14, wherein a pharmaceutical acceptable carrier and/or diluent comprises at least one hydrophilic polymeric compound selected from the group consisting of a gum, a cellulose ether, an acrylic resin, a carbohydrate carrier, talc, lactose, mannitol, glucose, water, gelatin, a protein-derived compound, polyvinyl pyrrolidone, magnesium stearate, and any combination thereof. Embodiment 16 is the composition of any one of embodiments 14 to 15, wherein the composition provides a lower concentration in the plasma of the subject at a dosage of about 0.5 mg/kg to 5 mg/kg. Embodiment 17 is the composition of any one of embodiments 1 to 16, wherein the composition is capable of decreasing the maximum mean glucose concentration in the plasma of said subject of from 200 mg/dL or more to about 160 mg/dL or less after administration of a dosage of 0.5 mg/kg to 5 mg/kg. Embodiment 18 is the composition of embodiment 17, wherein the glucose concentration in the plasma of the subject is 90 to 160 mg/dl, 85 mg/dL to 150 mg/dL, or 80 mg/dL to 100 mg/dL. Embodiment 19 is the composition of any one of embodiments 1 to 18, wherein the composition remains stable after being stored for one month, 6 months, 12 months, 18 months, or 24 months at room temperature. Embodiment 20 is the composition of any one of embodiments 1 to 19, wherein the composition comprises 0.001 wt. % or more, 0.1 wt. % or more, 10 wt. % or more or 99 wt. % or more of the ellagic dihydrate compound. Embodiment 21 is the composition of any one of embodiments 1 to 20, wherein the composition is formulated for oral administration. Embodiment 22 is the composition of embodiment 21, wherein the composition is provided as a powder, a tablet, a gel-cap, a bead, an edible tablet, a gelatin, a lotion, a transdermal patch, or a liquid solution. Embodiment 23 is the composition of any one of embodiments 21 to 22, wherein the composition is comprised in a solid nanoparticle, a lipid-containing nanoparticle, a lipid-based carrier, a sealed conduit, a straw, sealed bag, or any combination thereof. Embodiment 24 is the composition of any one of embodiments 1 to 23, wherein the composition is formulated for administration by injection. Embodiment 25 is a method of treating a subject comprising administering to the subject an effective amount of the composition as claimed in embodiment 1, wherein the ellagic acid dihydrate compound provides an increase in glucose uptake with a dose effect under in vitro conditions as compared to free ellagic acid. Embodiment 26 is the method of any one of embodiments 25 to 27, wherein the subject has been diagnosed with Type I diabetes, Type II diabetes, gestational diabetes, latent auto-immune diabetes of adulthood, insulin resistant diseases, prediabetes, clinical obesity, metabolic syndrome, liver disease, kidney disease, coronary heart disease, cognitive diseases, or diseases associated with advanced glycation end products. Embodiment 27 is the method of embodiment 26, wherein the advanced glycation end products diseases comprise atherosclerosis, chronic renal failure, dementia, or Alzheimer's. Embodiment 28 is the method of any one of embodiments 25 to 27, wherein administering the composition is part of a regime to prevent birth defects in pregnant women or women of childbearing age. Embodiment 29 is the method of any one of embodiments 25 to 28, wherein the compound is administered orally. Embodiment 30 is the method of any one of embodiments 25 to 29, wherein the composition provides a maximum mean glucose concentration in the plasma of said subject of 120 to 160 mg/dl, 80 mg/dL to 200 mg/dL, or 80 mg/dL to 150 mg/dL, or 90 to 100 mg/dL after administration of a dosage of 0.5 mg/kg to 5 mg/kg. Embodiment 31 is the method of any one of embodiments 25 to 31, wherein the subject is a mammal. Embodiment 32 is the method of embodiment 31, wherein the mammal is a mouse, rat, rabbit, cat, dog, pig, monkey, or ape. Embodiment 33 is the method of embodiment 32, wherein the subject is a human. Embodiment 34 is the method of any one of embodiments 25 to 33, wherein the subject experiences weight loss after 7 days, 14 days, 30 days, 1 year or more of administration of the composition. Embodiment 35 is a method of treating obesity or controlling body weight of a subject comprising administering to the subject an effective amount of the composition as claimed in embodiment 1, wherein the subject experiences weight loss after 7 days or more. Embodiment 36 is the method of embodiment 35, wherein the subject is administered between 0.005 and 100 mg, 0.05 and 75 mg, 0.1 and 50 mg, or 1 and 90 mg of the ellagic acid dihydrate compound/lb. of the subject daily. Embodiment 37 is the method of any one of embodiments 35 to 36, wherein the weight loss continues after 14 days or more, 30 days or more, or 1 year or more. Embodiment 38 is the method of any one of embodiments 35 to 37, wherein the composition provides loss of at least 5%, at least 8%, or at least 10% or more of the subject's body weight after 30 days of treatment. Embodiment 39 is the method of any one of embodiments 35 to 38, wherein the subject experiences an increase in cellular energy level. Embodiment 40 is a pharmaceutical composition formulated for administration to a human subject for the treatment of a hyperglycemic disorder, the composition comprising an ellagic acid dihydrate compound, wherein the composition provides over a period of time a reduction in a maximum mean glucose concentration in blood plasma. Embodiment 41 is a method for treating a hyperglycemic disorder comprising administrating to a subject the composition comprising an ellagic acid dihydrate compound as claimed in any one of embodiments 1 to 24, wherein the composition provides a reduction in a maximum mean glucose concentration in blood plasma over a period of time. Embodiment 42 is a pharmaceutical composition formulated for administration to a human subject for the treatment of a glucose metabolism disorder, the composition comprising an ellagic acid dihydrate compound, wherein the composition provides over a period of time a reduction in a maximum mean glucose concentration in blood plasma. Embodiment 43 is a method for treating a glucose metabolism disorder, the method comprising administrating a composition as claimed in any one of embodiments 1 to 24, wherein the composition provides over a period of time a reduction in a maximum mean glucose concentration in blood plasma. Embodiment 44 is a pharmaceutical composition formulated for administration to a subject for the treatment of obesity or control of body weight, the composition comprising an ellagic acid dihydrate compound. Embodiment 45 is the pharmaceutical composition of embodiment 44, wherein the composition is formulated to provide a weight loss in the subject of at least 5%, at least 8%, at least 10% or more, or at least 20% of body weight after 30 days of treatment. Embodiment 46 is the pharmaceutical composition of any one of embodiments 44 to 45, wherein the subject is administered from 0.001 to 100 mg of ellagic acid dihydrate compound/kg of the subject daily. Embodiment 47 is a pharmaceutical composition formulated for administration to a human subject for the treatment of a glucose metabolism disorder, the composition comprising an ellagic acid dihydrate compound derived from an extract of Punica granatum (pomegrante) having a purity of 95% or more. Embodiment 48 is a stable, pharmaceutical composition formulated for subcutaneous administration to a mammalian subject, said composition comprising ellagic acid dihydrate compound. Embodiment 49 is the stable, pharmaceutical composition of Embodiment 48, wherein the mammalian subject is a human subject. Embodiment 50 is the stable, pharmaceutical composition of embodiment 48, wherein the mammalian subject is a veterinary subject. Embodiment 51 is the stable, pharmaceutical composition of embodiment 48, wherein the mammalian subject is a canine or feline subject. Embodiment 52 is a pharmaceutical composition formulated for oral administration to a human subject for the treatment of hyperglycemia or controlling body weight, the composition comprising an ellagic acid dihydrate compound. Embodiment 53 is a method of treating a subject comprising administering to the subject an effective amount of an ellagic acid dihydrate compound wherein the ellagic acid dihydrate compound provides an increase in the ratio of AKT to phosphorylated AKT in a cell. Embodiment 54 is a method of treating a subject comprising administering to the subject an effective amount of an ellagic acid dihydrate compound wherein the ellagic acid dihydrate compound provides an increase in the ratio of p44/42 MAPK to phosphorylated p44/42 MAPK in a cell. Embodiment 55 is a method of treating a subject comprising administering to the subject an effective amount of an ellagic acid dihydrate compound wherein the ellagic acid dihydrate compound provides an increase in ruffling of filopodia at the surface of the cell. Embodiment 56 is a method of treating a subject comprising administering to the subject an effective amount of an ellagic acid dihydrate compound wherein the ellagic acid dihydrate compound provides an increase in GLUT4 concentration at the cellular membrane.

In some aspects of the invention, the composition may further comprise one or more pharmaceutically acceptable carriers or diluents. These carriers/diluents can be adjuvants, excipients, or vehicles such as preserving agents, fillers, disintegrating agents, wetting agents, emulsifiers, suspending agents, sweeteners, flavorings, fragrance, antibacterial agents, antifungal agents, lubricating agents, vitamins, polymers, siloxane containing compounds, essential oils, structuring agents, and dispensing agents. Each carrier is acceptable in the sense of being compatible with the other ingredients of the formulation and not injurious to the subject. In some aspects of the invention, the pharmaceutical acceptable carrier can include at least one hydrophilic polymeric compound selected from the group consisting of a gum, a cellulose ether, an acrylic resin, a carbohydrate carrier, talc, lactose, mannitol, glucose, water, gelatin, a protein-derived compound, polyvinyl pyrrolidone, magnesium stearate, and any combination thereof. Non-limiting examples of diluents/carriers are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

The stable compound can be stored for one month, 6 months, 12 months, 18 months, or 24 months at room temperature. In some aspects of the invention, the composition is formulated as a powder, a tablet, a gel-cap, a bead, an edible tablet, a gelatin, a lotion, a transdermal patch, or a liquid solution for oral administration. In some aspects of the invention, the formulated composition can be comprised in a solid nanoparticle, a lipid-containing nanoparticle, a lipid-based carrier, a sealed conduit, a straw, sealed bag, or any combination thereof. In other aspects of the invention, the composition can be formulated for administration by injection.

Kits that include the compositions of the present invention are also contemplated. In certain embodiments, the composition is comprised in a container. The container can be a bottle, dispenser, package, or a straw. The container can dispense a predetermined amount of the composition. In certain aspects, the compositions are dispensed as a pill, a tablet, a capsule, a transdermal patch, an edible chew, a cream, a lotion, a gel, spray, mist, dollop, a powder, or a liquid. The container can include indicia on its surface. The indicia can be a word, an abbreviation, a picture, or a symbol.

It is contemplated that any embodiment discussed in this specification can be implemented with respect to any method or composition of the invention, and vice versa. Furthermore, compositions of the invention can be used to achieve methods of the invention.

Also contemplated is a product that includes the composition of the present invention. In non-limiting aspects, the product can be a pharmaceutical product. The pharmaceutical product can be those described in other sections of this specification or those known to a person of skill in the art. Non-limiting examples of products include a pill, a tablet, an edible chew, a capsule, a cream, a lotion, a gel, a spray, a mist, a dissolving film, a transdermal patch, or a liquid, etc.

“Therapeutic agent” encompasses ellagic acid dihydrate compound. It also encompasses such compounds together with pharmaceutically acceptable salts thereof.

Useful salts are known to those skilled in the art and include salts with inorganic acids, organic acids, inorganic bases, or organic bases. Therapeutic agents useful in the present invention are those compounds that affect a desired, beneficial, and often pharmacological, effect upon administration to a human or an animal, whether alone or in combination with other pharmaceutical excipients or inert ingredients.

The term “ellagic acid dihydrate compound” refers to the ellagic acid dihydrate, analogues thereof, derivatives thereof, or salt forms of any analogue or derivative thereof.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

“Patient,” “subject,” or “individual” refers to a mammal (e.g., human, primate, dog, cat, bovine, ovine, porcine, equine, mouse, rate, hamster, rabbit, or guinea pig). In particular aspects, the patient, subject, or individual is a human.

“Inhibiting” or “reducing” or any variation of these terms includes any measurable decrease or complete inhibition to achieve a desired result.

“Effective” or “treating” or “preventing” or any variation of these terms means adequate to accomplish a desired, expected, or intended result.

“Analogue” and “analog,” when referring to a compound, refers to a modified compound wherein one or more atoms have been substituted by other atoms, or wherein one or more atoms have been deleted from the compound, or wherein one or more atoms have been added to the compound, or any combination of such modifications. Such addition, deletion or substitution of atoms can take place at any point, or multiple points, along the primary structure comprising the compound.

“Derivative,” in relation to a parent compound, refers to a chemically modified parent compound or an analogue thereof, wherein at least one substituent is not present in the parent compound or an analogue thereof. One such non-limiting example is a parent compound which has been covalently modified. Typical modifications are amides, carbohydrates, alkyl groups, acyl groups, esters, pegylations and the like.

A “therapeutically equivalent” drug is one that has essentially the same effect in the treatment of a disease or condition as one or more other drugs. A drug that is therapeutically equivalent may or may not be chemically equivalent, bioequivalent, or generically equivalent.

“Parenteral injection” refers to the administration of small molecule drugs via injection under or through one or more layers of skin or mucus membranes of an animal, such as a human.

“Bioavailability” refers to the extent to which the therapeutic agent, such as an ellagic acid dihydrate compound, is absorbed from the formulation.

“Systemic,” with respect to delivery or administration of a therapeutic agent, such as a ellagic acid dihydrate compound, to a subject, that therapeutic agent is detectable at a biologically significant level in the blood plasma of the subject.

“Controlled release” refers to the release of the therapeutic agent at such a rate that blood (e.g., plasma) concentrations are maintained within the therapeutic range, but below toxic concentrations over a period of time of about one hour or longer, preferably 12 hours or longer.

“Pharmaceutically acceptable carrier” refers to a pharmaceutically acceptable solvent, suspending agent or vehicle for delivering a drug compound of the present invention to a mammal such as an animal or human.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

The term “mammal” or “mammalian” includes murine (e.g., rats, mice) mammals, rabbits, cats, dogs, pigs, and primates (e.g., monkey, apes, humans). In particular aspects in the context of the present invention, the mammal can be a murine mammal or a human.

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%. Further, “substantially non-aqueous” refers to less than 5%, 4%, 3%, 2%, 1%, or less by weight or volume of water.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the transitional phase “consisting essentially of,” in one non-limiting aspect, a basic and novel characteristic of the compositions and methods disclosed in this specification includes the compositions' abilities to reduce or prevent obesity, Type 1 diabetes, Type II diabetes, gestational diabetes, latent auto-immune diabetes, prediabetes, metabolic syndrome, etc.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A depicts a model of a crystal structure of free ellagic acid.

FIG. 1B depicts a model of a crystal structure of ellagic acid dihydrate.

FIG. 2 depicts cell proliferation of MEFs after 48 hr exposure to ellagic acid dihydrate, vehicle, or no treatment.

FIG. 3 is a bar graph of cell membrane integrity of MEFs after 48 hr exposure to ellagic acid dihydrate, vehicle, or no treatment using propidium iodide (PI) labeling.

FIG. 4A is a graph of compound concentration (nMolar) versus 2-NBDG Signal in MEFs after exposure to ellagic acid dihydrate, insulin, or vehicle (Neg.) using 2-NBDG labeling.

FIG. 4B is a graph of compound concentration (nMolar) versus percent increase glucose uptake in MEFs after exposure to ellagic acid dihydrate or insulin over the vehicle control treatment using 2-NBDG labeling.

FIG. 5A is a graph of compound concentration (nMolar) versus 2-NBDG Signal in C2C12s after exposure to ellagic acid dihydrate, insulin, or vehicle (Neg.) using 2-NBDG labeling.

FIG. 5B is a graph of compound concentration (nMolar) versus percent increase glucose uptake in C2C12s after exposure to ellagic acid dihydrate or insulin over the vehicle control treatment using 2-NBDG labeling.

FIG. 5C is a graph of compound concentration (nMolar) versus percent increase glucose uptake in C2C12s after exposure to metformin, insulin and a second batch of ellagic acid dihydrate (ellagic acid dihydrate 2).

FIG. 6 is a graph of compound concentrations (nMolar) versus 2-NBDG Signal in MEFs using flow cytometry after exposure to ellagic acid dihydrate, ellagic acid, insulin, or vehicle (Neg.) using 2-NBDG labeling.

FIG. 7 is graphs of compound concentrations (nMolar) versus 2-NBDG Signal in MEFs using Operetta (20x) imaging after exposure to ellagic acid dihydrate, ellagic acid, insulin, or vehicle (Neg.) using 2-NBDG labeling.

FIG. 8 is a bar graph depicting the ratio of AKT to phosphorylated AKT (Phos AKT) in log2 scale in C2C12s after exposure to metformin, insulin and ellagic acid dihydrate relative to vehicle treated cells using immunocytochemistry labeling (anti-AKT, anti-Phos AKT) on a high-content imaging system.

FIG. 9 is a bar graph depicting the ratio of p44/42 MAPK to phosphorylated p44/42 MAPK (Phos p44/42 MAPK) in log2 scale in C2C12s after exposure to metformin, insulin and ellagic acid dihydrate relative to vehicle treated cells using immunocytochemistry labeling (anti-p44/42 MAPK, anti-Phos p44/42 MAPK) on a high-content imaging system (Operetta, Perkin Elmer).

### DETAILED DESCRIPTION

Without wishing to be bound by theory, it is believed that ellagic acid dihydrate compounds are capable of stimulating uptake of glucose. Further, it is believed that ellagic acid dihydrate compounds are capable of stimulating uptake of glucose under in vitro conditions. The uptake can be increased as the unit dosage of ellagic acid dihydrate is increased. Ellagic acid dihydrate compounds can stimulate glucose uptake to a greater extent than the use of free ellagic acid. Through the increased uptake of glucose, many health benefits can be realized. In some aspects, the uptake of glucose can result in weight reduction, increased brain function, stabilizing blood glycogen levels, increase cellular energy levels, and/or overall health benefits to a subject experiencing metabolic disorders. Diseases such as liver disease, kidney disease, and arterio-diseases can be affected be high levels of glucose in the blood plasma, thus an increase in uptake of glucose may result in improved liver function, kidney function and blood flow. Accordingly, it is possible to prepare a composition of ellagic acid dihydrate compounds that upon administration to a subject, the subject can experience an increase in uptake in glucose and/or increase in cellular energy levels, which results in an improvement in a subject's overall health. Further, it is possible to prepare a composition disclosed herein that can be used for the treatment or prevention of hyperglycemic disorders, obesity, and metabolic conditions and disorders and the related pathologies, conditions, and causes and symptoms of such disorders and conditions. Non-limiting examples of such disorders, conditions, pathologies, and symptoms include obesity, Type I diabetes, Type II diabetes, gestational diabetes, latent auto-immune diabetes, prediabetes, and metabolic syndrome.

### A. Ellagic Acid Dihydrate Compound

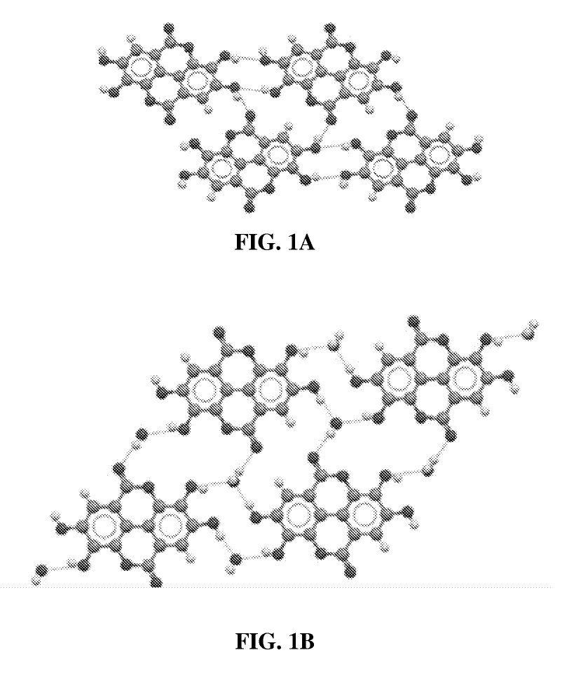

The composition of the present invention can include ellagic acid dihydrate compounds having the chemical structures (I) and/or (II). In a preferred embodiment, the ellagic acid dihydrate compound has a purity of greater than 98.0 wt. % and is a crystalline powder. Without wishing to be bound by theory it is believed that the ellagic acid dihydrate includes planar ellagic acid compounds interconnected by hydrogen bonds to water molecules giving rise to layers of molecules throughout the crystal, while free ellagic acid hydrogen bonds to itself to form a tightly packed crystal structure. Crystal structures of the free ellagic acid and ellagic acid dihydrate structure as reported by in the Cambridge Structural database are depicted in FIGS. 1A and 1B, respectively. Without wishing to be bound by theory, it is believed that these different crystal structures may contribute to the bioavailability of the ellagic acid dihydrate compound to the cells and/or subject.

The ellagic acid dihydrate derivatives and analogues can be made through known synthetic methods methylation and acylation reactions as described in U.S. Pat. No. 5,066,571 to Caufield, which is incorporated herein by reference. For example, ellagic acid dihydrate can be prepared by oxidative coupling of gallic acid (A) to form the dicarboxylic acid form of ellagic acid (B), followed by lactonization to form the desired compound (C) as shown in Scheme (I) and then crystallized in the presence of water. Derivatives of ellagic acid can be made using known reactions for amidization, esterification, etc.

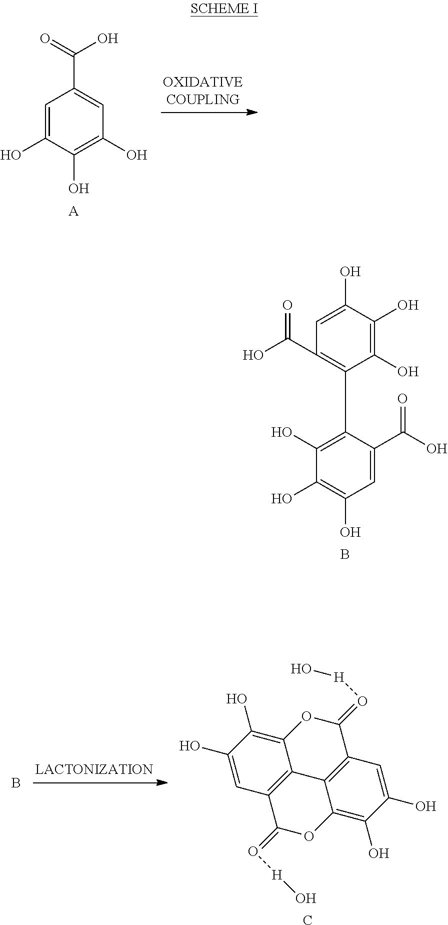

In some aspects of the invention, the ellagic acid dihydrate can be isolated from extracts of fruits and plants. Non-limiting examples of fruits include red raspberries, pomegranate, strawberries, and blueberries. Non-limiting examples of plants include extract of tree bark. Non-limiting examples of sources of tree bark include Anisophyllea dichostyla; Elaeocarpus parvifolius; Eucalyptus globulus; Platycarya strobilacea; Punica granatum; a species of the genus Castanea; a species of the genus Terminalia; and a species of the genus Quercus. In some aspects of the invention, by-products of the paper industry may be used as a source for ellagic acid and/or ellagitannins. Plants and fruits contain the natural product ellagitannins. The ellagitannins can be extracted from the plants and fruits using known extraction methods, such as contacting the tree sawdust with an alcohol at ambient temperature with agitation, followed by filtration to obtain the extract. The extract can include ellagitannins, ellagic acid and other products. The extract can be subjected to acid hydrolysis conditions to hydrolyze the ellagitannins (D) to the open acid form of ellagic acid (B), followed by subsequent lactonization under acid or basic conditions to produce the ellagic acid dihydrate (C) as shown in Scheme II. Ellagic acid dihydrate is commercially available from many chemical suppliers. Non-limiting examples of suppliers are Sigma-Aldrich (USA) and TCI Fine Chemicals (China/Japan).

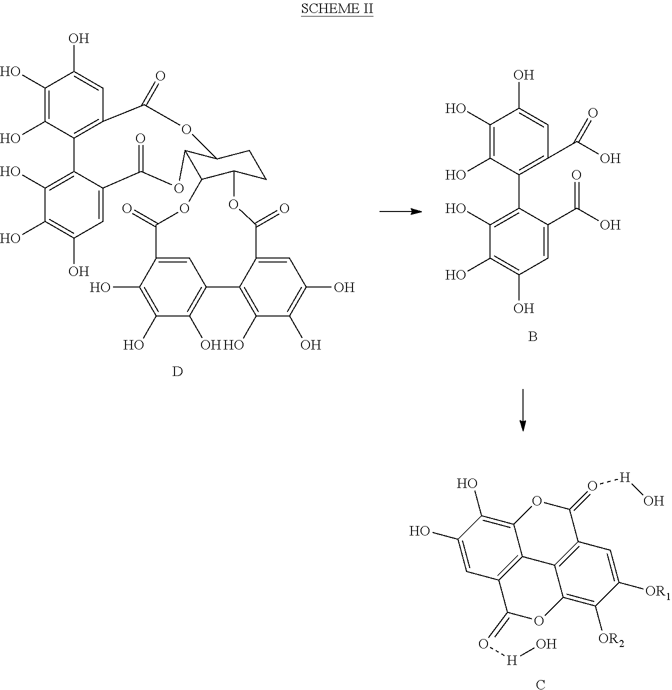

### B. Amounts of Ingredients

It is contemplated that the compositions of the present invention can include any amount of the ingredients discussed in this specification. The compositions can also include any number of combinations of additional ingredients described throughout this specification (e.g., stabilizers, fillers, pharmaceutically acceptable salts, and/or additional pharmaceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### C. Additional Components

The ellagic acid dihydrate compound of the present invention can be formulated into any suitable composition form for administration to a human or non-human animal patient.

The composition may consist of the ellagic acid dihydrate alone or may include the ellagic acid dihydrate and any suitable additional component, such as one or more pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the patient.

1. Excipients

Excipients employed in the compositions of the present invention can be solids, semi-solids, liquids or combinations thereof. Preferably, the excipients are solids. Compositions of the invention containing excipients can be prepared by any known technique that includes, for example, admixing an excipient with the ellagic acid dihydrate. A pharmaceutical composition of the invention contains a desired amount of the ellagic acid dihydrate per dose unit and, if intended for oral administration, can be in the form, for example, of a tablet, a caplet, a pill, a hard or soft capsule, a lozenge, a cachet, a dispensable powder, granules, a suspension, an elixir, a dispersion, or any other form reasonably adapted for such administration. If intended for parenteral administration, it can be in the form, for example, of a suspension or transdermal patch. If intended for rectal administration, it can be in the form, for example, of a suppository. Presently preferred are oral dosage forms that are discrete dose units each containing a predetermined amount of the ellagic acid dihydrate such as tablets or capsules.

2. Carriers/Diluents

Suitable carriers or diluents illustratively include, but are not limited to, either individually or in combination, lactose, including anhydrous lactose and lactose monohydrate; starches, including directly compressible starch and hydrolyzed starches (e.g., Celutab™ and Emdex™), mannitol, sorbitol, xylitol, dextrose (e.g., Cerelose™ 2000) and dextrose monohydrate, dibasic calcium phosphate dihydrate, sucrose-based diluents, confectioner's sugar, monobasic calcium sulfate monohydrate, calcium sulfate dihydrate, granular calcium lactate trihydrate, dextrates, inositol, hydrolyzed cereal solids, amylose, celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC), calcium carbonate, glycine, clay, bentonite, block co-polymers, polyvinylpyrrolidone, and the like. Such carriers or diluents, if present, constitute in total about 5% to about 99.999%, about 10% to about 85%, and 20% to about 80%, of the total weight of the composition. The carrier, carriers, diluent, or diluents selected preferably exhibit suitable flow properties and, where tablets are desired, compressibility.

3. Disintegrant

Compositions of the invention optionally can include one or more pharmaceutically acceptable disintegrants as excipients, particularly for tablet formulations. Suitable disintegrants include, but are not limited to, either individually or in combination, starches, including sodium starch glycolate and pregelatinized corn starches, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums. Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

4. Binders

The compositions of the present invention can include binding agents or adhesives particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a co-crystal of the present invention once the salt has been dissolved in a solution. Suitable binding agents and adhesives include, but are not limited to, either individually or in combination, acacia; tragacanth, sucrose, gelatin, glucose, starches such as, but not limited to, pregelatinized starches, celluloses such as, but not limited to, methylcellulose and carmellose sodium, alginic acid and salts of alginic acid; magnesium aluminum silicate, PEG, guar gum, polysaccharide acids, bentonites, povidone, polymethacrylates, HPMC, hydroxypropylcellulose, and ethylcellulose. Such binding agents and/or adhesives, if present, constitute in total about 0.5% to about 25%, preferably about 0.75% to about 15%, and more preferably about 1% to about 10%, of the total weight of the pharmaceutical composition. Many of the binding agents are polymers comprising amide, ester, ether, alcohol or ketone groups and, as such, can be included in pharmaceutical compositions of the present invention. Polyvinylpyrrolidones is an non-limiting example of a binder used for slow release tablets. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units' ratios in a given polymer affects properties and performance.

5. Wetting Agents

Wetting agents can be used in the compositions of the present invention. Wetting agent can be selected to maintain the crystal in close association with water, a condition that is believed to improve bioavailability of the composition. Such wetting agents can also be useful in solubilizing or increasing the solubility of crystals. Surfactants can be used as wetting agents. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, poloxamers (polyoxyethylene and polyoxypropylene block copolymers), polyoxyethylene fatty acid glycerides and oils, for example polyoxyethylene (8) caprylic/capric mono- and diglycerides, polyoxyethylene (35) castor oil and polyoxyethylene (40) hydrogenated castor oil, polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the pharmaceutical composition.

6. Lubricants

Lubricants can be included in the compositions of the present invention. Suitable lubricants include, but are not limited to, either individually or in combination, glyceryl behapate, stearic acid and salts thereof, including magnesium, calcium and sodium stearates; hydrogenated vegetable oils, colloidal silica, talc, waxes, boric acid, sodium benzoate, sodium acetate, sodium fumarate, sodium chloride, DL-leucine, PEG (e.g., Carbowax™ 4000 and Carbowax™ 6000 of the Dow Chemical Company), sodium oleate, sodium lauryl sulfate, and magnesium lauryl sulfate. Such lubricants, if present, constitute in total about 0.1% to about 10%, preferably about 0.2% to about 8%, and more preferably about 0.25% to about 5%, of the total weight of the pharmaceutical composition.

7. Other Agents

Surfactant, emulsifier, or effervescent agents can be used in the compositions. Emulsifying agents can be used to help solubilize the ingredients within a soft gelatin capsule. Non-limiting examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

### D. Vehicles

Various delivery systems are known in the art and can be used to administer a therapeutic agent or composition of the invention, e.g., encapsulation in liposomes, microparticles, microcapsules, receptor-mediated endocytosis and the like. Methods of administration include, but are not limited to, parenteral, intra-arterial, intramuscular, intravenous, intranasal, and oral routes. The pharmaceutical compositions can be provided in the form of tablets, lozenges, granules, capsules, pills, ampoule, suppositories or aerosol form. The pharmaceutical compositions can also be provided in the form of suspensions, solutions, and emulsions of the active ingredient in aqueous or non-aqueous diluents, syrups, granulates or powders.

### E. Formulation and Administration

The composition may, for example, be a pharmaceutical composition (medicament). Pharmaceutical compositions according to the present invention include formulations suitable for oral or parenteral routes. Non-limiting examples of specific routes include intradermal, subcutaneous, intramuscular, intravenous, local injection, rectal, intranasal inhalation, insufflation, topical (including transdermal, buccal and sublingual), vaginal, parenteral (including subcutaneous, intramuscular, intravenous and intradermal) and pulmonary administration. The formulations can conveniently be presented in unit dosage form and can be prepared by any methods well known in the art of pharmacy. Such methods include the step of bringing into association the active ingredient (for example, ellagic acid dihydrate) with the carrier which constitutes one or more accessory ingredients. In general, the formulations are prepared by uniformly and intimately bringing into association the active ingredient with a suitable carrier, such as liquid carriers or finely divided solid carriers or both, and then if necessary shaping the product. Formulations of the subject invention suitable for oral administration can be presented as discrete units such as capsules, cachets or tablets, each containing a predetermined amount of the active ingredient, or as an oil-in-water liquid emulsion, water-in-oil liquid emulsion, or as a supplement within an aqueous solution, for example, a tea. The active ingredient can also be presented as bolus, electuary, or paste. Useful injectable preparations include sterile suspensions, solutions or emulsions of the ellagic acid dihydrate compound compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives. Alternatively, the injectable formulation can be provided in powder form for reconstitution with a suitable vehicle, including but not limited to sterile pyrogen free water, buffer, dextrose solution, etc., before use. To this end, the ellagic acid dihydrate compound compositions can be dried by any art-known technique, such as lyophilization, and reconstituted prior to use.

Formulations suitable for topical administration in the mouth include lozenges comprising the active ingredient in a flavored basis, usually sucrose and acacia or tragacanth, pastilles that include the active ingredient in an inert basis such as gelatin and glycerin, or sucrose and acacia, mouthwashes that include the active ingredient in a suitable liquid carrier, and chocolate comprising the active ingredients.

Formulations suitable for topical administration according to the subject invention can be formulated as an ointment, cream, suspension, lotion, powder, solution, paste, gel, spray, aerosol or oil. Alternatively, a formulation can comprise a patch or a dressing such as a bandage or adhesive plaster impregnated with active ingredients, and optionally one or more excipients or diluents. Topical formulations preferably comprise compounds that facilitate absorption of the active ingredients through the skin and into the bloodstream.

Formulations suitable for intranasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for administration by nebulizer, include aqueous or oily solutions of the agent. Formulations preferably can include compounds that facilitate absorption of the active ingredients through the skin and into the bloodstream.

Formulations suitable for parenteral administration include aqueous and non-aqueous isotonic sterile injection solutions which can contain antioxidants, buffers, bacteriostats and solutes which render the formulation isotonic with the blood of the intended recipient; and aqueous and non-aqueous sterile suspensions which can include suspending agents and thickening agents, and liposomes or other microparticulate systems which are designed to target the compound to blood components or one or more organs. The formulations can be presented in unit-dose or multi-dose or multi-dose sealed containers, such as for example, ampoules and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules and tablets of the kind previously described.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

For buccal administration, the compositions can take the form of tablets or lozenges formulated in conventional manner.

For rectal and vaginal routes of administration, the ellagic acid dihydrate compound compositions can be formulated as solutions (for retention enemas) suppositories or ointments containing conventional suppository bases such as cocoa butter or other glycerides.

For nasal administration or administration by inhalation or insufflation, the ellagic acid dihydrate compound compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

For prolonged delivery, the ellagic acid dihydrate compound compositions can be formulated as a depot preparation for administration by implantation or intramuscular injection. The ellagic acid dihydrate compound compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the ellagic acid dihydrate compound compositions for percutaneous absorption, can be used. To this end, permeation enhancers can be used to facilitate transdermal penetration of the ellagic acid dihydrate compound compositions. Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other delivery systems can be employed. Liposomes and emulsions are well-known examples of delivery vehicles that can be used to deliver the ellagic acid dihydrate compound compositions. Certain organic solvents such as dimethylsulfoxide (DMSO) can also be employed, although usually at the cost of greater toxicity.

It should be understood that in addition to the ingredients particularly mentioned above, the formulations useful in the present invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

In one embodiment, the pharmaceutical compositions of the invention can be administered locally to the area in need of treatment; such local administration can be achieved, for example, by local infusion, by injection, or by means of a catheter. In another embodiment, a compound or composition of the invention is administered in a manner so as to achieve peak concentrations of the active compound at sites of the disease. Peak concentrations at disease sites can be achieved, for example, by intravenously injecting of the agent, optionally in saline, or orally administering, for example, a tablet, capsule or syrup containing the active ingredient.

### F. Other Pharmaceutical Agents

Pharmaceutical formulations of the invention can be administered simultaneously or sequentially with other drugs or biologically active agents. Examples include, but are not limited to, antioxidants, free radical scavenging agents, analgesics, anesthetics, anorectals, antihistamines, anti-inflammatory agents including non-steroidal anti-inflammatory drugs, antibiotics, antifungals, antivirals, antimicrobials, anti-cancer actives, antineoplastics, biologically active proteins and peptides, enzymes, hemostatics, steroids including hormones and corticosteroids, etc.

### G. Therapeutic Methods and Dosage

Preferred unit dosage formulations are those containing a daily dose or unit, daily sub dose, or an appropriate fraction thereof, of an agent. Therapeutic amounts can be empirically determined and will vary with the pathology being treated, the subject being treated, and the efficacy and toxicity of the agent. Similarly, suitable dosage formulations and methods of administering the agents can be readily determined by those of ordinary skill in the art.

In some embodiments, a therapeutic method of the present invention can include treating a disease, condition, or disorder by administering to a subject having such disease or condition a stable formulation as described herein in an amount effective to treat the disease, condition, or disorder. In some embodiments, the subject is administered a stable formulation comprising ellagic acid dihydrate compounds. The disease, condition, or disorder can be caused by hyperglycemia or obesity. Further, the disease, condition, or disorder can be Type 1 diabetes, Type II diabetes, gestational diabetes, latent auto-immune diabetes, prediabetes, or metabolic syndrome and related diseases, conditions, and disorders. For prophylactic administration, the composition can be administered to a patient at risk of developing one of the previously described conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art. In some aspects of the invention, total dosage amounts of a ellagic acid dihydrate compound composition will typically be in the range of from about 0.0001 or 0.001 or 0.01 mg/kg/day to about 100 mg/kg/day, but may be higher or lower, depending upon, among other factors, the activity of the components, its bioavailability, the mode of administration and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. Skilled artisans will be able to optimize effective local dosages without undue experimentation. In some embodiments, the compositions can be administered in conjunction with other compound known to effect weight loss. For example, the compounds can be given together with compounds known to burn fat.

### H. Kits

In another aspect of the present invention, kits for treating a disease, condition or disorder as described herein. For instance, compositions of the present invention can be included in a kit. A kit can include a container. Containers can include a bottle, a metal tube, a laminate tube, a plastic tube, a dispenser, a straw, a pressurized container, a barrier container, a package, a compartment, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a predetermined amount of the composition. In other embodiments, the container can be squeezed (e.g., metal, laminate, or plastic tube) to dispense a desired amount of the composition. The composition can be dispensed as a spray, an aerosol, a liquid, a fluid, a semi-solid, or a solid. In a preferred embodiments, the composition is dispensed as a free flowing powder or particles. The containers can have spray, pump, or squeeze mechanisms. A kit can also include instructions for employing the kit components as well the use of any other compositions included in the container. Instructions can include an explanation of how to apply, use, and maintain the compositions. The compositions can, if desired, be presented in a pack or dispenser device, which can contain one or more unit dosage forms containing the ellagic acid dihydrate compound compositions. The pack can, for example, comprise metal or plastic foil, such as a blister pack. The pack or dispenser device can be accompanied by instructions for administration.

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Formulation for Examples 2 Through 7

General Procedure. For Examples 2 through 7, all test compounds of interest were initially dissolved in 100% DMSO. Estimated saturation occurred at approximately 4 mM for ellagic acid dihydrate. The saturated solution was diluted 1:10 in 1× PBS; creating a 100× stock solution of 400 μM ellagic acid dihydrate. Working concentrations (1×), 4 μM, and more dilute concentrations of ellagic acid dihydrate were created by 1:10 dilutions into 0.1% DMSO. The resulting six dilutions ranged from 4 μM to 0.04 nM. Dose curves were analyzed using either Harmony software on the Operetta (Perkin Elmer) or Excel (Microsoft) for flow cytometry, which was performed on a BD Accuri (BD Biosciences).

### Example 2

### Ellagic Acid Dihydrate Interaction with Glucose

Ellagic acid dihydrate was analyzed for direct interaction with glucose. A glucose oxidase based glucometer was used to measure serum glucose. Ellagic acid dihydrate was incubated with serum at room temperature for five minutes or overnight at 37° C. Upon testing the mixture, no inhibition of the glucose oxidase reaction was observed. Ellagic acid dihydrate is not believed to directly interact with glucose.

### Example 3

### Cytotoxicity

The cytotoxicity of ellagic acid dihydrate in in vitro cell culture was evaluated. Cellular assays were performed using mouse embryonic fibroblasts (MEFs). Cells were unexposed (No Treatment) or exposed to varying concentrations of ellagic acid dihydrate (4 μM-0.04 nM) or vehicle (0.1% DMSO) for 48 hrs. All assays were run in triplicate.

Cell proliferation rates were determined using automated cell counting of cells after 48 hr exposure to ellagic acid dihydrate in complete medium (DMEM w/10% FBS). Cell were then washed thrice with 1× PBS and incubated with Accutase (Invitrogen) for 10 min at 37° C., neutralized with 10% FBS in 1× PBS, and analyzed by flow cytometry. Cell concentrations were determined by cell counting (500-1000) on a flow cytometer (Accuri C6, BD Biosciences). The concentration of cells reflects the impact of ellagic acid dihydrate on the cell population numbers (cell death and cell proliferation).

Loss of cellular membrane integrity is an indication of cell death. Cell membrane integrity/cell toxicity was determined by adding propidium iodide (PI) to suspended cells prior to flow cytometry. PI is a red-fluorescent DNA stain that is excluded by live-healthy cells but will enter dead cells in a population. The presence of PI in 500-1000 cells per treatment group was determined by flow cytometry (Accuri C6, BD Biosciences). Cell with disrupted membranes, associated with cell death, are expected to be positively labeled with PI.

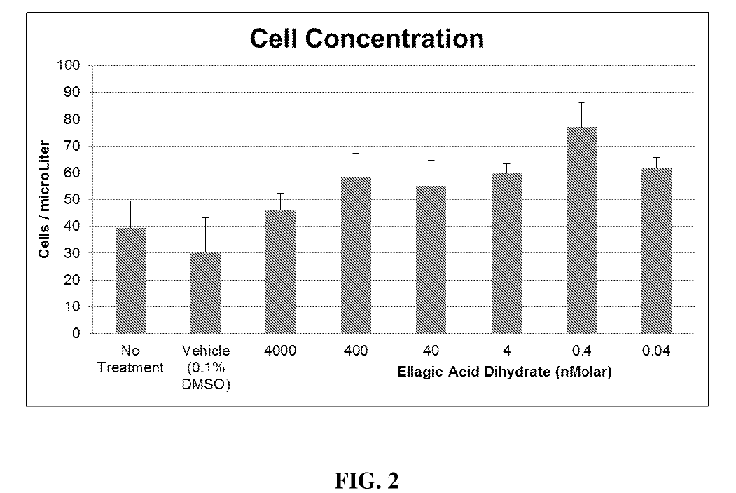

Results: Cell proliferation of MEFs after 48 hr exposure to ellagic acid dihydrate showed a trend toward increasing cell proliferation that appears dose related. See FIG. 2. However, the changes in cell proliferation produced were not statistically significant (P>0.05). Thus, an overall, yet non-significant, increase in cell concentration was observed with increasing concentration of ellagic acid dihydrate. These data indicate no negative impacts on cell population numbers upon exposure to ellagic acid dihydrate.

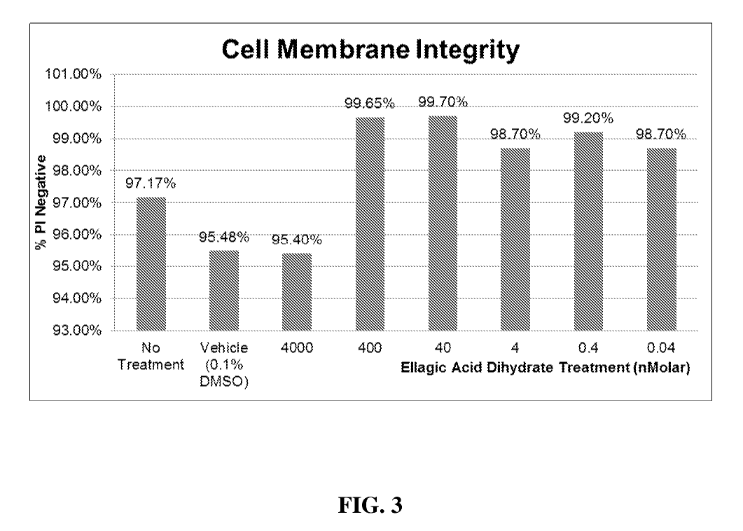

Cell membrane integrity (% cells negative for PI) of MEFs after 48 hr exposure to ellagic acid dihydrate was increased relative to the vehicle or untreated cells for treatments 400 nM-0.4 nM. FIG. 3. A decrease in integrity was observed relative to vehicle at 4000 nM (95.48%, 95.40%, respectively). The changes in cell death/membrane integrity were not statistically significant (P>0.05). FIG. 3. These data indicated no negative impacts on cell membrane integrity upon exposure to ellagic acid dihydrate.

### Example 4

### Glucose Uptake in Ellagic Acid Dihydrate Insulin and Metformin Treatments

Glucose uptake in ellagic acid dihydrate treated cell culture was measured and compared to glucose uptake under insulin or metformin treatment. Cellular assays were performed using mouse myoblast C2C12 cells and MEF cells. Cells were unexposed (No Treatment) or exposed to varying concentrations of ellagic acid dihydrate (4 μM-0.04 nM) or vehicle (0.1% DMSO) or similar concentrations of insulin or metformin.

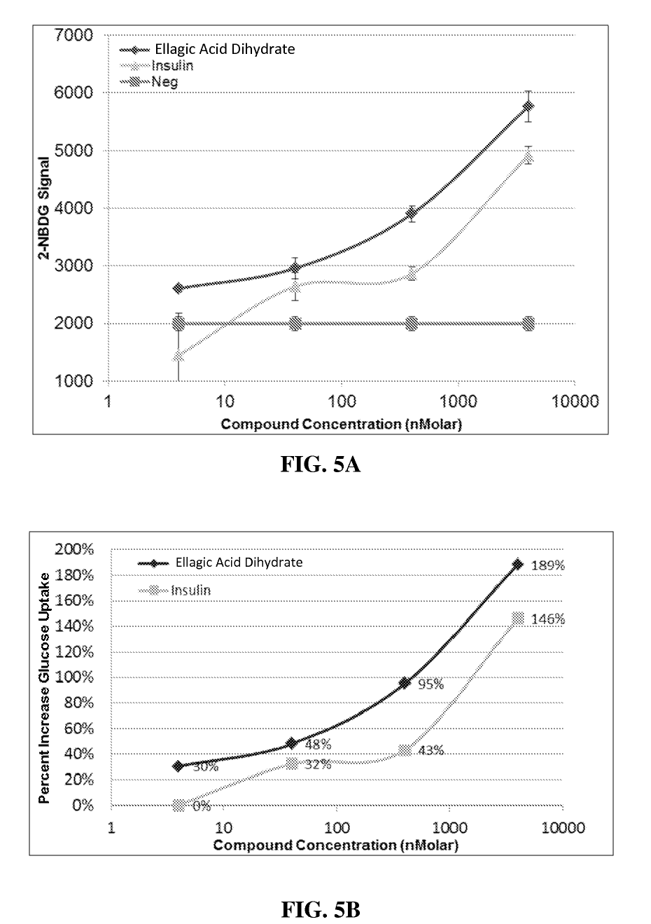

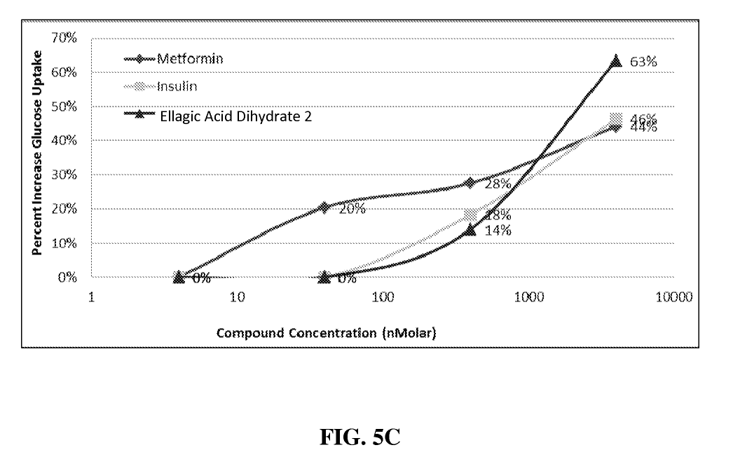

Glucose uptake was determined in MEFs using fluorescently labeled glucose, 2-(N-(7-Nitrobenz-2-oxa-1,3-diazol-4-yl)Amino)-2-Deoxyglucose (2-NBDG) and measured either on a high-content imaging system (Operetta, Perkin Elmer) or by flow cytometry (500 to 1000 cells) (Accuri C6, BD Biosciences). Increased glucose uptake was indicated as increasing signal of 2-NBDG (488 nM) in cells. Glucose uptake assays were carried out in a final incubation volume of 50 μl consisting of, 2-NBDG), (100 μM) in 1× Phosphate Buffered Saline w/Ca+, Mg+ (PBS++). The same buffer was used for washing cells. Insulin was used as a positive control. All testing compounds were prepared in 10% DMSO and diluted 1:100 for a final concentration of 0.1% DMSO (vehicle). Cells were culture to 70-90% confluence and then washed twice with PBS++. Cells were incubated for 30 minutes at 37° C. in PBS++. Nuclear stain, HCS CellMask stain (blue, Invitrogen), and/or testing compounds (ellagic acid dihydrate, insulin, or vehicle) were added. Cells were incubated for 30 min at 37° C. Cells were washed twice with PBS++and incubated with 2-NBDG for 10 minutes. Cells were washed thrice with PBS++and then imaged in PBS++. Alternatively, the final PBS wash was removed and cells were incubated with Accutase (Invitrogen) for 10 min at 37° C., neutralized with 10% FBS in 1× PBS, and analyzed by flow cytometry. An additional experiment was performed to measure glucose uptake in C2C12 cells exposed to metformin, insulin and ellagic acid dihydrate 2 (batch 2). FIG. 5C.

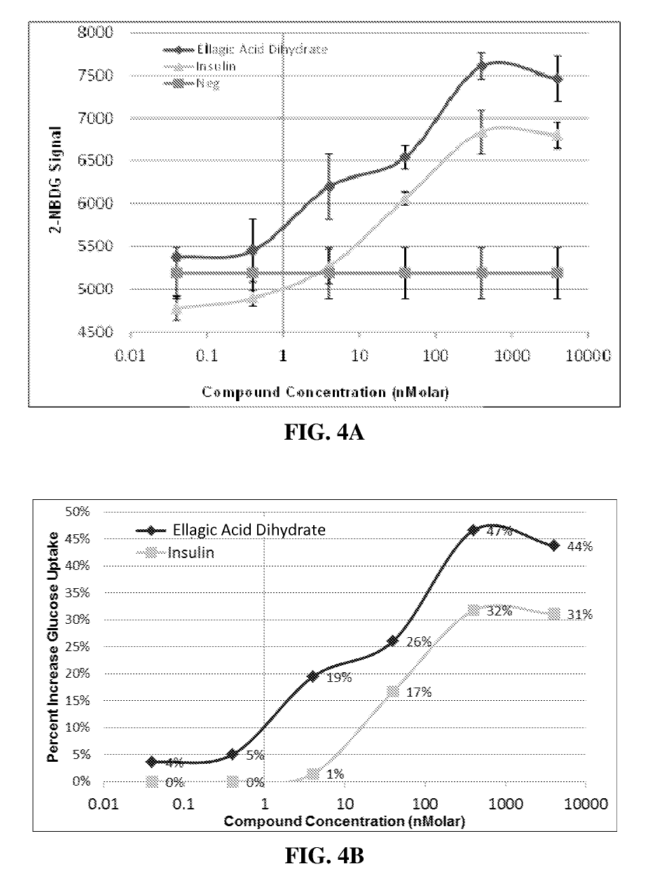

Results: Insulin and ellagic acid dihydrate showed a dose effect for increasing glucose uptake in both cell lines using flow cytometry. FIGS. 4 and 5 (FIGS. 4A, 4B and 5A, 5B) show the 2-NBDG uptake curves for test compounds. FIGS. 4A and 5A indicate the fluorescent signal of glucose (2-NBDG) taken up by the cell. FIGS. 4B and 5B indicate the relative glucose uptake as a percent change relative to vehicle treated (0.1% DMSO) cells. Insulin produced a significant increase in glucose uptake relative to the vehicle in both cell lines. FIGS. 4A and 5A. The impact of ellagic acid dihydrate on glucose uptake (2-NBDG signal) in both cell lines was significantly greater than vehicle treated (Neg, 0.1% DMSO). FIGS. 4A and 5A. At most concentrations tested, ellagic acid dihydrate induced significantly more glucose uptake relative to insulin in both fibroblasts and myoblasts. FIGS. 4B and 5B. For example, ellagic acid dihydrate and insulin increased glucose uptake by 47% and 32%, respectively, at 400 nM exposures in fibroblasts. FIG. 4B. The increase in glucose uptake produced by ellagic acid dihydrate and insulin was ˜4× greater in myoblasts (See, FIGS. 5A and 5B) relative to fibroblasts (See, FIGS. 4A and 4B). Ellagic acid dihydrate also showed a greater increase in glucose uptake than metformin at 4000 nm. FIG. 5C.

Further, glucose uptake was determined in MEFs using 2-NBDG labeling of cells on a high-content imaging system (Operetta, Perkin Elmer). Insulin and ellagic acid dihydrate showed an increase in glucose uptake as indicated by increasing signal of 2-NBDG (488 nM, green) in cells. Insulin and ellagic acid dihydrate showed a dose effect for increasing glucose uptake in MEFs. The impact of ellagic acid dihydrate on glucose uptake (2-NBDG signal) was significantly greater than vehicle treated (Neg, 0.1% DMSO) or Insulin treated cells (data not show). The data paralleled results shown by flow cytometry.

The increased uptake of glucose in multiple cell types by ellagic acid dihydrate confirms that this compound is capable of stimulating glucose uptake in cultured myoblasts and fibroblast cells. At equal concentrations, the uptake induced by ellagic acid dihydrate exceeded the uptake induced by insulin in both cell lines tested. This effect was significant at 4 nM-4 μM.

### Example 5

### Comparison of Glucose Uptake with Insulin and Ellagic Acid

Glucose uptake in ellagic acid dihydrate treated cell culture was measured and compared to glucose uptake under insulin or ellagic acid treatment. Cellular assays were performed using MEF cells as outlined in Example 4 above except that cells were unexposed (No Treatment) or exposed to varying concentrations of ellagic acid dihydrate (4 μM-0.04 nM), vehicle (0.1% DMSO), or similar concentrations of insulin or ellagic acid. Flow cytometry results were run in triplicate with 1000 cells per batch.

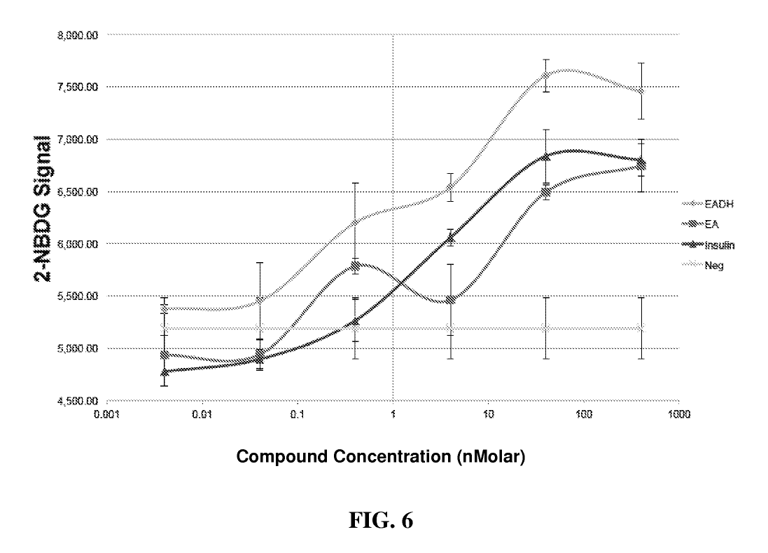

Results: Glucose uptake determined in MEFs using 2-NBDG labeling of cells in a flow cytometry showed an increase dose effect for increasing glucose uptake as indicated by increasing signal of 2-NBDG (488 nM, green) in cells for all treatments in comparison to vehicle (Neg.). FIG. 6. Ellagic acid dihydrate (EADH) treatment showed a significantly greater increase in glucose uptake than that seen in ellagic acid (EA) or insulin treatment. FIG. 6. Thus, surprisingly, ellagic acid dihydrate was shown to increase glucose uptake significantly greater than ellagic acid or insulin treatment. This suggests that ellagic acid dihydrate may be a more effective treatment for diseases and conditions related to higher glucose levels or decreased glucose uptake or metabolism than either insulin or ellagic acid.

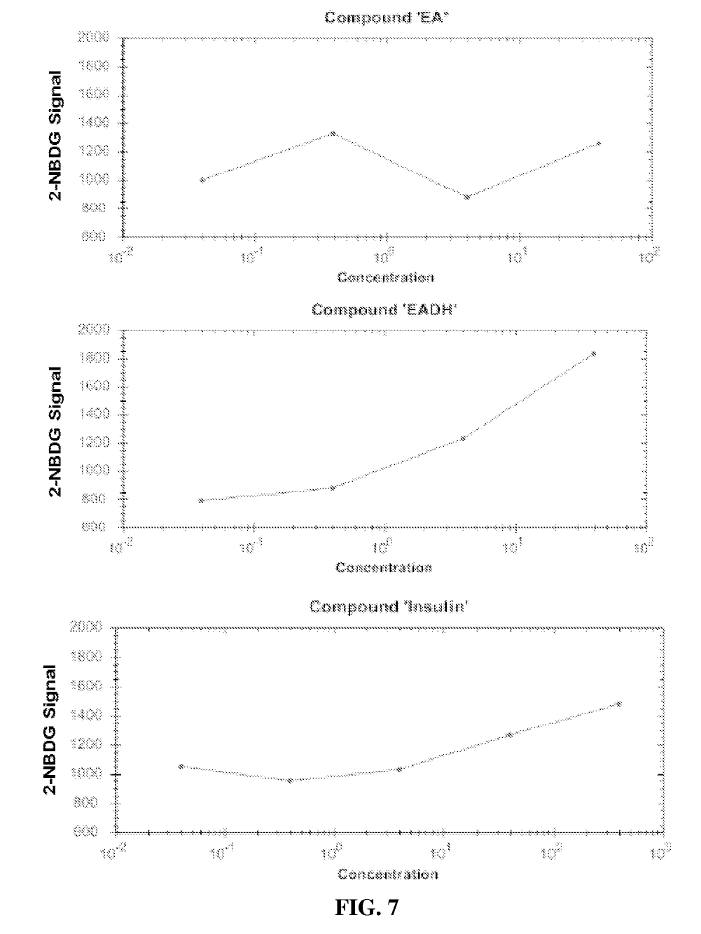

Glucose uptake determined in MEFs using 2-NBDG labeling of cells on a high-content imaging system (Operetta, Perkin Elmer) showed an increase in glucose uptake as indicated by increasing signal of 2-NBDG (488 nM, green) in cells for all treatments in comparison to vehicle (Neg.). FIG. 7. Ellagic acid dihydrate (EADH) and insulin showed a dose effect for increasing glucose uptake in MEFs, but no such dose effect was seen in cells treated with ellagic acid (EA). FIG. 7. Again, this suggests that ellagic acid dihydrate may be a more effective treatment for diseases and conditions related to higher glucose levels or decreased glucose uptake or metabolism than ellagic acid.

### Example 6

### Localization of Glucose Transporter, GLUT4

Induction of glucose transporter, GLUT-4, in ellagic acid dihydrate treated cell cultures was studied. Cellular assays were performed using mouse myoblast C2C12 cells. The cells were infected with a fusion glucose transporter 4 green fluorescent protein (GLUT4-GFP). This protein allows the visualization of GLUT4 by imaging the GFP under fluorescent time-lapse microscopy. Frames were compiled to produce time-lapse images of cellular changes with no treatment or treatment with insulin.

The localization of glucose transporter (GLUT4-GFP) was performed in final incubation volume of 505 μL, consisting of 500 μL of Hank's Buffer (Invitrogen) and 5 μl of tested compounds. Insulin was used as positive controls and vehicle (0.1% DMSO) as the negative control. All test compounds were prepared in 10% DMSO and diluted 1:100 for a final concentration of 0.1% DMSO (vehicle). The cells were rinsed in Hank's Buffer thrice and then incubated in Hank's Buffer at 37° C. for one hour. The test compounds were added (ellagic acid dihydrate, insulin, or vehicle) and the cells were imaged using laser-confocal microscopy (Leica TCS SP5 X) with FITC fluorescent filters to determine changes in GLUT4-GFP localization. Time-lapse images (TIF) were collected and post-processed using Image-J (NIH) for video construction.

Results: Changes in cells due to insulin or ellagic acid dihydrate exposure was seen. The fluorescent signal (GLUT4-GFP) was predominately located around the nucleus prior to the insulin exposure. Exposure to insulin (4000 nM) produced a decrease in GLUT4-GFP around the cell nucleus and a parallel increase of movement at the membrane. Further, insulin treatment induced rapid production of numerous projections (ruffling of filopodia) at the surface of the cell. The increase in membrane and GLUT4-GFP activity was directly associated with an increase in glucose uptake by insulin. Exposure to ellagic acid dihydrate (4000 nM) also produced a decrease in nuclear GLUT4-GFP and rapid ruffling of filopodia at the surface of the cell. These increase in membrane and GLUT4-GFP activity were directly associated with an increase in glucose uptake by ellagic acid dihydrate. These observations paralleled those seen with insulin; however, this process appeared slightly delayed in the ellagic acid dihydrate treated cells. There was no indication of changes in nuclear localization of GLUT4-GFP or ruffling of filopodia in any time-lapse imaging of untreated or vehicle treated cells (data not shown).

### Example 7

### Comparison of Activation of AKT and MAPK Pathways with Ellagic Acid Dihydrate, Insulin and Metformin

Stimulation of AKT pathways and MAPK pathways in ellagic acid dihydrate, metformin, and insulin treated cell cultures was performed. Insulin stimulates glucose uptake into cells by increasing GLUT4 activation, and AKT, specifically phos-AKT, signaling alters GLUT4 translocation. MAPK has also been suggested as being involved in the GLUT4 activation pathway. Cellular assays were performed using mouse myoblast C2C12 cells. Relative protein levels of AKT:phos AKT and p44/42 MAPK:phos p44/42 MAPK were determined.

Serum starved cells (1 hr) were treated for 30 minutes with each of the tested compounds. Ratios of phosphorylated to non-phosphorylated proteins were determined using immunocytochemistry labeling (anti-AKT, anti-Phos AKT; or anti-p44/42 MAPK, anti-Phos p44/42 MAPK) on a high-content imaging system (Operetta, Perkin Elmer) using the fluorescent signal of anti-rabbit Alexa 647. Increased phosphorylation is indicated as decrease in signal of AKT:Phos AKT ratio (log2 scale) and p44/42 MAPK:Phos p44/42 MAPK ratio (log2 scale) relative to vehicle treated cells.

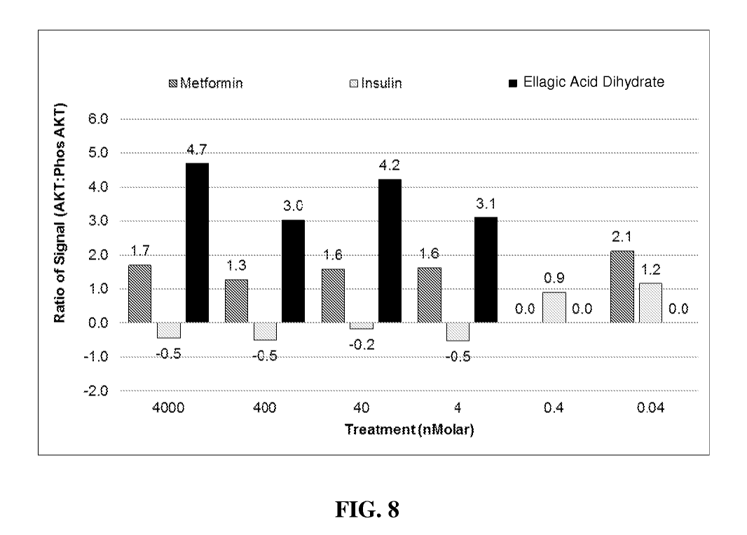

Results: Treatment of cells with insulin showed an increase in Phos-AKT (−0.5 ratio) with treatment (4 nM-4 μM). FIG. 8. Thus, insulin increased phosphorylation of AKT. Ellagic acid dihydrate shows an increase in AKT relative to Phos-AKT signal (4 nM-4 μM). FIG. 8. Thus, ellagic acid dihydrate increased AKT relative to phosphorylation of AKT. Metformin showed an increase in AKT relative to phosphorylation of AKT. FIG. 8.

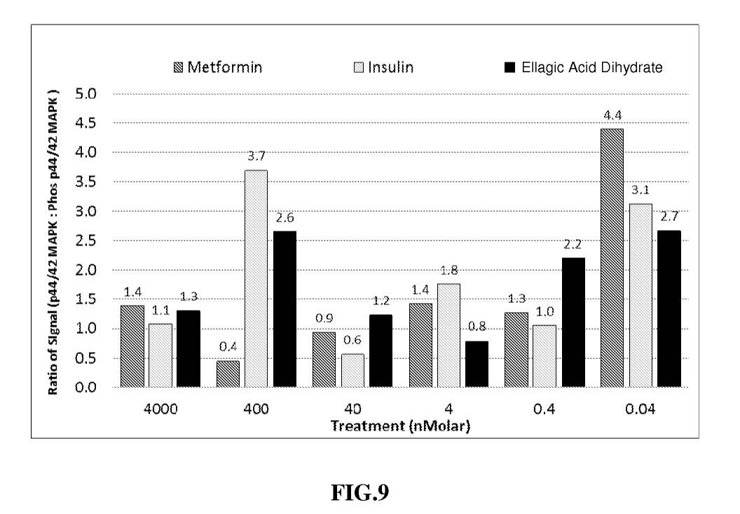

Insulin and ellagic acid dihydrate show an increase in p44/42 MAPK with treatment (400 nM, 0.04 nM). FIG. 9. Metformin increased the ratio of p44/42 MAPK at 0.04 nM treatment. FIG. 9. No treatments were observed to increase the ratio of phosphorylated p44/42 MAPK relative to p44/42 MAPK signal. FIG. 9.

### Example 8

### Assays for Determining Weight Loss and Modified Glucose Regulation in Mice and Data for Ellagic Acid Treatment

Weight loss and blood glucose in mice treated with ellagic acid was determined and weight loss and blood glucose in mice treated with ellagic acid dihydrate may be determined by the following procedure.

Mice are treated daily for thirty days with ellagic acid or control in the form of a supplemental pill homogenized and suspended in 25% water solution of Cremophor EL. The mice are treated via gavage using the human equivalent dose (LD) of 122.4 mg pill/kg of subject (40.6 mg ellagic acid/kg of the subject), 10× higher dose (HD) of 1224 mg pill/kg of the subject (406 mg ellagic acid/kg of the subject), or control vehicle (25% water solution of Cremophor EL). The LD and HD dose amounts are calculated based on the following assumptions: a human dose is one 603 mg (200 mg ellagic acid) pill/day, taken orally; the average weight of a person is 60 kg, thus, the average dose for a human equals approximately 9.95 mg of the pill (3.3 mg ellagic acid)/kg daily; a mouse equivalent dose requires 12.3 times the human dose per kg. The mice are weighed and treated daily, and blood glucose level are measured every two days for a total one month period.

Results: An ellagic acid treatment study was performed in female SWV mice treated daily with ellagic acid in the form of supplemental pill using the human equivalent dose (LD) and 10× higher dose (HD) for thirty days. The LD treated mice lost on average 8.9% of body weight and the HD treated mice lost on average 10.4% of body weight. At the same time the control dams gained 3.2% of the initial body weight. Normal levels of blood glucose were seen in all treatment compared to the control groups. However, it is predicted, based on the in vitro data reported in Example 5 of ellagic acid dihydrate in comparison to ellagic acid, that mice treated with ellagic acid dihydrate will have a greater decrease in weight loss and may have some reduction in blood glucose levels compared to mice treated with ellagic acid.

### Example 9

### Assays for Determining Weight Loss and Modified Glucose Regulation in Mice with STZ Induced Diabetes and Data for Ellagic Acid Treatment

Weight loss and blood glucose in diabetes induced mice treated with ellagic acid was determined and weight loss and blood glucose in mice treated with ellagic acid dihydrate may be determined by the following procedures.

Induction of Diabetes: Diabetes is induced in mice by intraperitoneal injection with Streptozotocin (STZ 40 mg/kg) daily for five days. The mice are considered hyperglycemic when the glucose level of each dam is above 200 mg/dL. The mice are fasted for 4 hours before STZ injection and glucose measurement.

Treatment: The mice are treated daily for thirty days with ellagic acid or control in the form of a supplemental pill homogenized and suspended in 25% water solution of Cremophor EL. The mice are treated for one month via gavage using the human equivalent dose of 122.4 mg pill/kg of subject (40.6 mg ellagic acid/kg of the subject), the human equivalent does of 40.6 mg ellagic acid/kg of the subject using 98% pure ellagic acid, or control vehicle (25% water solution of Cremophor EL). The human equivalent dose is calculated as described in Example 8. Treatments would start two weeks after the last STZ injection in which hyperglycemia is confirmed in the diabetic mice. The mice would be weighed daily and glucose levels would be measured in fasted animals every five days.

Results: An ellagic acid treatment study was performed in female SWV mice with STZ induced diabetes. The mice were treated daily with ellagic acid in the form of supplemental pill or 98% pure substance (human equivalent dose) for thirty days. The mice treated with the supplemental pill saw a lost on average of 18.9% of body weight and the mice treated with 98% pure substance (human equivalent dose) lost on average 11.7% of body weight. At the same time the control dams lost 2.2% of the initial body weight. The treated mice were hyperglycemic (glucose level above 200 mg/dL) for the entire period of the treatment, no significant differences between the mean glucose level values were seen between the treated and the control mice. However, it is predicted, based on the in vitro data reported in Example 5 of ellagic acid dihydrate in comparison to ellagic acid, that mice treated with ellagic acid dihydrate will have a greater decrease in weight loss and will have lower mean glucose level values than mice treated with ellagic acid.

### Example 10

### Assays for Determining Activity Level and Observations for Ellagic Acid Treatment

The Applicant has noted that mice treated with ellagic acid seem to have increased activity levels. It is predicted based on the comparative in vitro reported in Example 5 using ellagic acid dihydrate in comparison to ellagic acid, that mice treated with ellagic acid dihydrate will show a significantly greater activity level than mice treated with ellagic acid. Increased activity in a mouse may suggest increased cellular energy levels such as increased ATP concentration, phosphocreatine levels, etc. Activity levels in mice treated with ellagic acid dihydrate may be quantified by several procedures, such as those outlined in Turri, M. et al.; QTL Analysis Identifies Multiple Behavioral Dimensions in Ethological Tests of Anxiety in Laboratory Mice; Current Biology; 2001; 11(10): 725-734. Two examples of procedures taught therein follows.

Gross Locomotor Activity in Home Cage: Mice are caged singly several days prior to home cage activity monitoring and subsequent testing. Activity in home cages are measured during the first 2 hours of the dark cycle on two consecutive days. Two infrared photo emitter-detector pairs, located outside the clear plastic home cage that divide the cage into three equal areas, are used to record gross locomotor activity on the floor of the home cage as the animal breaks the photo beams. The beam-break activity score consists of the square root of the number of beam breaks for each detector summed across the two test days.

Magnitude of Activity in Home Cage: Mice are caged singly several days prior to home cage activity monitoring and subsequent testing. Activity in home cages are measured during the first 2 hours of the dark cycle on two consecutive days. An infrared motion detector located above the top of the home cage is employed to converts all motion sensed within the home cage into arbitrary units, based on the magnitude of activity detected. Mice showing high levels of activity restricted to one area of the home cage can obtain relatively high activity scores with motion detection monitoring as opposed to the gross locomotor activity test above.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1.-56. (canceled)

57. A method of treating a subject, the method comprising administering to the subject an effective amount of a pharmaceutical composition comprising an ellagic acid dihydrate compound, wherein the ellagic acid dihydrate compound provides an increase in glucose uptake per unit dose as compared to an increase in glucose uptake provided by free ellagic acid.

58. The method of treating a subject of claim 57, wherein the subject has been diagnosed with Type I diabetes, Type II diabetes, gestational diabetes, latent auto-immune diabetes of adulthood, an insulin resistant disease, prediabetes, clinical obesity, a metabolic syndrome, a liver disease, a kidney disease, a coronary heart disease, a cognitive disease, a hyperglycemic disorder, a glucose metabolism disorder, being overweight, or a disease associated with advanced glycation end products, or is a pregnant woman or woman of childbearing age with a risk of having birth defects in her fetus.

59. The method of claim 57, wherein the ellagic acid dihydrate compound assists in:
a) increasing glucose uptake, reducing a maximum mean glucose concentration in blood plasma;
b) increasing GLUT4 concentration at a cellular membrane, increasing in ruffling of filopodia at the surface of a cell;
c) increasing a ratio of p44/42 MAPK to phosphorylated p44/42 MAPK in a cell;
d) increasing a ratio of AKT to phosphorylated AKT in a cell;
e) increasing a cellular energy level,
f) controlling the body weight of the subject; or
g) combinations thereof.

60. The method of claim 57, wherein the pharmaceutical composition provides a maximum mean glucose concentration in blood plasma of said subject of 120 to 160 mg/dl after administration of a dosage of 0.5 mg/kg to 5 mg/kg of the ellagic acid dihydrate compound.

61. The method of claim 57, wherein the ellagic acid dihydrate compound is represented by the formula: 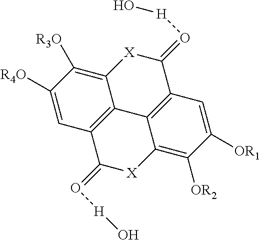
where R₁, R₂, R₃, and R₄ are each independently selected from a hydrogen (H), a C₁-C₂₀ alkyl or ether group, an acyl group, an aryl group, an aralkyl group, an amide, an amino acid, and a heterocyclic group, and X is a heteroatom.

62. The method of claim 57, wherein the ellagic acid dihydrate compound consists essentially of:

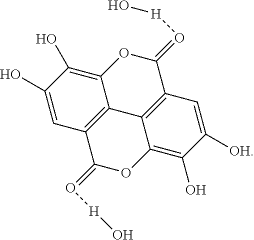

63. The method of claim 57, wherein pharmaceutical composition is administered orally and/or by injection.

64. The method of claim 57, wherein the subject is administered between 0.005 and 100 mg of the ellagic acid dihydrate compound/lb. of the subject daily and wherein the ellagic acid dihydrate compound is capable of providing an increase in glucose uptake per unit dose under in vitro conditions as compared to an increase in glucose uptake provided by free ellagic acid.

65. The method of claim 57, wherein the pharmaceutical composition further comprises a pharmaceutical acceptable carrier and/or diluent comprising a carbohydrate carrier, talc, lactose, mannitol, glucose, water, gelatin, a protein-derived compound, polyvinyl pyrrolidone, magnesium stearate, at least one hydrophilic polymeric compound selected from a gum, a cellulose ether, and an acrylic resin, and any combination thereof.

66. The method of claim 57, wherein the method decreases the concentration of glucose in blood plasma of the subject at a dosage of the composition of 0.5 mg/kg to 5 mg/kg.

67. The method of claim 57, wherein a maximum mean glucose concentration in blood plasma of the subject is decreased from 200 mg/dL or more to about 160 mg/dL or less after administration of a dosage of 0.5 mg/kg to 5 mg/kg of the pharmaceutical composition.

68. The method of claim 57, wherein the pharmaceutical composition is provided as a powder, a tablet, a gel-cap, a bead, an edible tablet, a gelatin, a lotion, a transdermal patch, or a liquid solution.

69. A pharmaceutical composition formulated for administration to a subject, the pharmaceutical composition comprising an ellagic acid dihydrate compound, wherein the compound is capable of providing an increase in glucose uptake per unit dose as compared to an increase in glucose uptake provided by free ellagic acid.

70. The pharmaceutical composition of claim 69, wherein the ellagic acid dihydrate compound is represented by the formula: 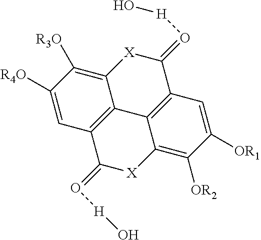
where R₁, R₂, R₃, and R₄ are each independently selected from a hydrogen (H), a C₁-C₂₀ alkyl or ether group, an acyl group, an aryl group, an aralkyl group, an amide, an amino acid, and a heterocyclic group, and X is a heteroatom.

71. The pharmaceutical composition of claim 69, wherein the ellagic acid dihydrate compound consists essentially of:

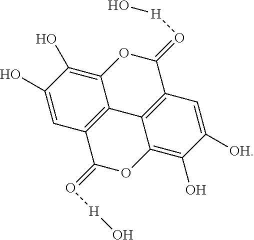

72. The pharmaceutical composition of claim 69, wherein the pharmaceutical composition is provided as an edible and/or injectable composition.

73. The pharmaceutical composition of claim 69, wherein the compound of the pharmaceutical composition is comprised in a powder, a tablet, a gel-cap, a bead, an edible tablet, a gelatin, a lotion, a transdermal patch, a liquid solution, a sealed bag, a solid nanoparticle, a lipid-containing nanoparticle, a lipid-based carrier, a sealed conduit, a straw, a tablet, a bottle, a sterile bag, or any combination thereof.

74. The pharmaceutical composition of claim 69, wherein the pharmaceutical composition is formulated for subcutaneous administration to a mammalian subject.

75. A pharmaceutical composition formulated for oral and/or injection administration to a human subject for the treatment of a hyperglycemic disorder, the pharmaceutical composition comprising an extract of a plant, the extract comprising an ellagic acid dihydrate compound being capable of providing an increase in glucose uptake as compared to free ellagic acid.

76. The pharmaceutical composition of claim 75, wherein the extract comprises 75% or more of the ellagic acid dihydrate compound.
